(19) United States
(12) Patent Application Publication
(10) Pub. No.: US 2003/0021836 A1
(43) Pub. Date: Jan. 30, 2003

(54) Magnesium di-potassium EDTA complex and method of administration

(76) Inventor: Spencer Feldman, Makawao, HI (US)

Correspondence Address: DARBY & DARBY P.C., P. O. BOX 5257, NEW YORK, NY, 10150-5257, US

(21) Appl. No.: 10/189,330

(22) Filed: Jul. 3, 2002

Related U.S. Application Data
(63) Continuation-in-part of application No. 10/124,763, filed on Apr. 19, 2002.
(60) Provisional application No. 60/285,546, filed on Apr. 20, 2001.

(51) Int. Cl. A61K 9/70; A61K 31/195

(52) U.S. Cl. 424/449; 514/566

(57) ABSTRACT

A composition containing an EDTA complex is provided. More specifically, the EDTA complex is a magnesium di-potassium EDTA complex. The complex may be administered to a patient in several forms and may contain a controlled release agent which will release the EDTA complex over a period of several hours.

This application is a continuation-in-part of application Ser. No. 10/124,763, filed Apr. 19, 2002, which in turn claims priority under 35 U.S.C. §119 from provisional patent application serial No. 60/285,546, filed Apr. 20, 2001, which is hereby incorporated by reference in its entirety.

### FIELD OF THE INVENTION

The present invention relates to a composition containing a chelating agent, more particularly to an EDTA complex.

### BACKGROUND OF THE INVENTION

Chelating agents are well known organic compounds that are capable of forming complexes of multivalent metal ions. Chelation therapy, involving the administration of EDTA (ethylene diamine tetraacetic acid) complexes for removing arterial calcium plaque, or for removing heavy metals such as lead has been extensively employed. For example, EDTA has been used to remove high levels of lead from the bloodstream of those who have been exposed to lead paint. Intravenous injection of chelators has been widely used for the treatment of atherosclerosis. Intravenous administration may take several hours per session, and several sessions per month for many months. Such a treatment schedule is inconvenient to the patient and health care provider.

Oral chelation therapy is also known as a more convenient method. However, when administered orally, a lower percentage of the EDTA complex is actually absorbed by the bloodstream.

It is further known that EDTA may be administered in a suppository form. Bennett U.S. Pat. No. 5,602,180 relates to a method of administering a disodium EDTA complex in the form of a controlled release suppository. U.S. Pat. No. 5,602,180 is hereby incorporated into this specification by reference.

Disodium EDTA has the disadvantage of raising sodium levels. Sodium stimulates the sympathetic system, causing, among other effects, an increase in blood pressure, pulse pressure, and heart rate. These affects are undesirable in the patient suffering from atherosclerosis, a condition for which EDTA therapy is often prescribed. As sodium increases pulse pressure, the amplitude of the cyclic changes in lumen size and shape are increased. These cyclic changes may lead to the disruption of plaque and subsequent myocardial infarction. See Valentin Fuster, MD, “The Vulnerable Atherosclerotic Plaque” (American Heart Association).

### SUMMARY OF THE INVENTION

The present invention provides an alternative to the intravenous injection of EDTA chelating agents. A method of administering EDTA complexes includes forming a composition containing a magnesium di-potassium EDTA complex. The complex may also contain a controlled-release agent, which, when used, will release the complex over a period of several hours.

### DETAILED DESCRIPTION OF THE INVENTION

According to this invention, a composition is formed of magnesium di-potassium EDTA complex and a matrix, which may or may not contain a time release agent. The composition may be administered to supplement magnesium and/or potassium levels and to treat arterial plaque and heavy metal toxicity.

The magnesium di-potassium EDTA complex may be obtained from Fluka of Switzerland as EDTA dipotassium magnesium salt monohydrate or EDTA dipotassium magnesium salt (product No. 40694). It can also be in the anhydrous form.

Compositions for use in accordance with the present invention may be formulated in a conventional manner using one or more physiologically acceptable carriers or excipients. Depending on the route of administration, the active substance may be coated in a material to protect the compound from the action of enzymes, acids and other natural conditions that may inactivate the compound. The compound may be formulated for oral, intranasal, sublingual, rectal or vaginal administration, or for administration transdermally.

For oral administration, the compositions may take the form of, for example, tablets, pills, capsules, or powders, prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. Tablets or pills can be coated or otherwise prepared so as to form a unit dosage form that has delayed and/or sustained action, such as controlled release and delayed release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the lower part of the digestive tract or to be delayed in release. As another example, the tablet or pill may comprise an inner dosage component of the magnesium di-sodium EDTA complex and an outer dosage component of an enteric component. As EDTA is often poorly absorbed as a result of being denatured by stomach acid, the enteric coating will allow the complex to be absorbed in the lower part of the digestive tract.

Biodegradable polymers for controlling the release of the active agents include, but are not limited to, polylactic acid, polyepsilon caprolactone, polyhydroxybutyric acid, polyorthoesters, polyacetals, polydihydropyrans, polycyanoacrylates and crosslinked or amphipathic block copolymers of hydrogels.

A transdermal dosage form may also be used. Transdermal forms may be a diffusion transdermal system (transdermal patch) using either a fluid reservoir or a drug-in-adhesive matrix system. Other transdermal dosage forms include, but are not limited to, topical gels, lotions, creams, ointments, transmucosal systems and devices, iontophoretic (electrical diffusion) delivery systems, and forms to be added to bath water, such as bath gels, oils powders or salts. Transdermal dosage forms may be used for delayed release and sustained release of the active agents of the present invention.

The compounds may also be formulated in the form of suppositories. Choosing suitable carrier materials, and suitable matrix ingredients, are within the skill of the artisan in this field. Representative prior U.S. patents include U.S. Pat. Nos. 4,265,875, 4,292,300, 4,406,883, 5,151,434, 5,188,840, 5,215,758, 5,352,455, and 5,393,528. The foregoing patents are hereby incorporated into this specification by reference. Such suppositories may include controlled release agents. The release-controlling agents, and their concentrations, should be chosen so that release occurs within the body over a one to four hour period after the suppository is administered. Such suppositories may be formulated for rectal or vaginal administration. The suppository is molded in a common shape from a waxy material in which the active ingredients have been dissolved or suspended. The base material may comprise cocoa butter, glycerin, glyceryl, monopalmitate, glyceryl monostearate, hydrogenated coconut oil fatty acids and hydrogenated palm kernel oil fatty acids or polyethylene glycol.

The precise dose of the constructs to be employed in the formulation depends on the route of administration, and the nature of the patient's symptoms and disorders, and should be decided according to the judgment of the practitioner and each patient's circumstances according to standard clinical techniques. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. Clinically significant attenuation or amelioration means perceptible to the patient and/or to the physician.

In addition to avoiding the disadvantages of disodium EDTA, the invention provides other advantages. Magnesium suppresses the sympathetic system and potassium stimulates the parasympathetic system. Thus the complex of the present invention may lower blood pressure, pulse and coronary blood flow. The magnesium di-potassium EDTA complex can also be used as a form of magnesium or potassium supplementation

What is claimed is:

1. A method of administering a magnesium di-potassium EDTA complex to a patient, comprising forming a transdermal dosage form containing magnesium di-potassium EDTA and administering said dosage form to the patient.

2. The method of claim 1 wherein the dosage form is a cream, gel, lotion or ointment.

3. The method of claim 1 wherein the dosage form is a sustained release formulation.

4. The method of claim 1 wherein the dosage form is a transdermal patch.

5. The method of claim 1 wherein the dosage form is a bath gel, bath oil, bath powder or bath salts.

6. A method of administering a magnesium di-potassium EDTA complex to a patient, comprising forming a sublingual dosage form containing magnesium d-potassium EDTA and administering said dosage form to the patient.
